SEQUENCE LISTING (2 sequences)

SEQ ID NO: 1 - DNA, 20 bp, Artificial Sequence
  synthetic primer 1
gagtcaacggatttggtcgt

SEQ ID NO: 2 - DNA, 20 bp, Artificial Sequence
  synthetic primer 2
ttgattttggagggatctcg

(19) United States
(12) Patent Application Publication
(10) Pub. No.: US 2010/0081176 A1
(43) Pub. Date: Apr. 1, 2010

(54) ENZYME-CONTAINING CAPSULES AND NUCLEIC ACID AMPLIFICATION KITS

(75) Inventor: Ken Takubo, Shizuoka (JP)

Correspondence Address: Townsend and Townsend and Crew LLP, Two Embarcadero Center, 8th Floor, San Francisco, CA, 94111, US

(73) Assignee: Beckman Coulter, Inc., Brea, CA (US)

(21) Appl. No.: 12/625,485

(22) Filed: Nov. 24, 2009

Related U.S. Application Data
(63) Continuation of application No. PCT/JP08/59375, filed on May 21, 2008.

(30) Foreign Application Priority Data
May 25, 2007 (JP) 2007-139143

(51) Int. Cl. C12P 19/34; C12N 9/96

(52) U.S. Cl. 435/91.2; 435/188

(57) ABSTRACT

An object of the present invention is to provide an enzyme-containing capsule which is employed for deactivating, from a sample, the proteins in the sample and then amplifying a nucleic acid in the sample, a kit for the amplification of nucleic acid which comprises the enzyme-containing capsules, and a method for the amplification of nucleic acid which employs the enzyme-containing capsules. The present invention provides an enzyme-containing capsule which is characterized in that the capsule has a melting point of 60 to 95° C., comprises a non-proteinous material as the envelope component, and contains a heat-resistant enzyme in the inside of the capsule; a kit for the amplification of nucleic acid, comprising the above-described enzyme-containing capsules and a proteolytic enzyme; and a method for the amplification of nucleic acid, employing the above-described enzyme-containing capsules.

### CROSS-REFERENCE TO RELATED APPLICATIONS

This application is a continuation of International Application No. PCT/JP2008/059375, filed May 21, 2008, which claims benefit of Japanese Patent Application No. 2007-139143, filed May 25, 2007; the disclosures of each are herein incorporated by reference.

### TECHNICAL FIELD

The present invention relates to an enzyme-containing capsule which is employed for amplifying, from a sample containing proteins and nucleic acids, a nucleic acid in the sample after deactivating the proteins in the sample, a kit for the amplification of a nucleic acid which comprises the enzyme-containing capsules, and a method for the amplification of a nucleic acid which employs the enzyme-containing capsules.

### BACKGROUND ART

With the recent progress in genetic engineering technology and molecular biology, analysis of nucleic acids contained in samples has come to be widely performed in the field not only of academic research, but also of medical treatment. For example, analysis of nucleic acids, such as genomic DNAs and mRNAs, in biological samples is performed for diagnosis of genetic diseases, cancers, infections, lifestyle-related diseases, and others. Such analysis is often performed by amplifying a target nucleic acid to be analyzed, since nucleic acids contained in biological samples are usually in very small amounts.

In biological samples, there are contained a variety of substances, including proteins, which can be factors inhibiting amplification reactions of nucleic acid. Among these proteins in particular are many proteins which have activities of degrading nucleic acids or inhibiting enzymes and others which are employed for nucleic acid amplification, and thus it is preferable that these proteins in biological samples be deactivated or removed, in order to amplify a target nucleic acid with high accuracy and high sensitivity. Therefore, amplification of nucleic acids is usually performed using biological samples on which pre-treatments, such as extraction of nucleic acids, have been done.

A variety of methods are disclosed for these pre-treatments. As methods of pre-treatment by which nucleic acids are extracted, for example, methods of extraction with phenol, methods of extraction using silica-gel particle columns, and others are disclosed. Here, methods of extraction with phenol refer to ones in which DNAs are extracted by (a) adding a surfactant to a biological sample to lyse cells, followed by digesting proteins with proteinase K, (b) removing the proteins by an extraction procedure with phenol, and (c) adding ethanol, thereby precipitating the DNAs (see, for example, Non-Patent Document 1). Methods of extraction using silica-gel particle columns refer to ones in which nucleic acids are extracted by (a) adding a surfactant to a biological sample to lyse cells, followed by digesting proteins with proteinase K, (b) passing the pre-treated biological sample through a silica-gel particle column, thereby allowing nucleic acids to be adsorbed on the silica-gel particles, and (c) then eluting the nucleic acids from the column with an eluent. Methods using silica-gel particle columns as described above are widely employed, for example, by means of commercially available kits, and fully automated machines for extracting nucleic acids are also commercially available, which use the above-mentioned silica-gel particles as magnetic particles.

There are also known methods of pre-treatment in which unlike the above-described methods, nucleic acids are not extracted, and which are carried out by adding an agent for neutralizing substances in biological samples that inhibit nucleic acid amplification, heating, and other treatments. Such methods include, for example, ones by which a polyamine is added into samples (see, for example, Patent Document 1), dithiothreitol is added (see, for example, Patent Document 2), sulfated polysaccharide is added (see, for example, Patent Document 3), a polyamine, dithiothreitol, and sulfated polysaccharide are added (see, for example, Patent Document 4), albumin is added (see, for example, Patent Document 5), a polyhydric alcohol and/or ammonium sulfate are/is added (see, for example, Patent Document 6), and a polymeric compound having a repeated structure containing anions (polyanion) and/or an insoluble polymer thereof are/is added (see, for example, Patent Document 7). In addition, there are known methods by which before performing the synthesis of a nucleic acid, a reaction solution for gene amplification to which a sample has been added is subjected to temperatures at which the thermal stability of heat-resistant enzyme is retained, for example, 70 to 90° C., for a period of 5 to 20 minutes (see, for example, Patent Document 8), methods by which PCR is performed under conditions of pHs which are higher than those which have been conventionally employed in many cases, that is, in a reaction solution having a pH of 8.9 or higher under conditions at a temperature of 25° C. (see, for example, Patent Document 9), and others.

In addition, as methods of pre-treatment in cases where amplification of nucleic acids is performed using RNA as the template, for example, ones by which the degradation of RNA and inhibition of amplification reactions are suppressed by adjusting the pH of solutions of lysed tissues and cells to 2.5 to 5, followed by adding to the solutions a chaotropic salt which interacts with substances inhibiting amplification reactions are included (see, for example, Patent Document 10). Other methods are also known, for example, boiling methods by which biological samples are simply subjected to boiling treatments (see, for example, Non-Patent Document 1).

[Non-Patent Document 1] Protein, Nucleic Acid, and Enzyme, Kyoritsu Shuppan Co. Ltd., 1996, vol. 41, No. 5, pages 453-456
Patent Document 1: Japanese Unexamined Patent Application, First Publication No. Hei 6-277061

### Patent Document 2: Japanese Unexamined Patent Application, First Publication No. 2000-93175

### Patent Document 3: Japanese Unexamined Patent Application, First Publication No. 2000-93176

### Patent Document 4: Japanese Unexamined Patent Application, First Publication No. 2001-8680

### Patent Document 5: Japanese Unexamined Patent Application, First Publication No. 2001-8685

### Patent Document 6: Japanese Unexamined Patent Application, First Publication No. 2000-352982

### Patent Document 7: Japanese Unexamined Patent Application, First Publication No. 2005-323617

Patent Document 8: Japanese Unexamined Patent Application, First Publication No. Hei 11-113573

### Patent Document 9: Japanese Unexamined Patent Application, First Publication No. 2003-174878

### Patent Document 10: Japanese Unexamined Patent Application, First Publication No. 2001-8680

### Patent Document 11: Japanese Patent No. 3,313,358

### Patent Document 12: Japanese Patent No. 3,433,929

### Patent Document 13: Published Japanese Translation No. 2000-515397 of the PCT Application

### Patent Document 14: Published Japanese Translation No. 2004-315466 of the PCT Application

### DISCLOSURE OF THE INVENTION

### Problems to be Solved by the Invention

It cannot be said that the above-described methods of extraction with phenol are preferable methods, since phenol itself is a poisonous substance. The above-described methods of extraction using silica-gel particle columns, on the other hand, result in the degradation and deactivation of proteins which are main substances in biological samples that inhibit nucleic acid amplification, and thus are highly preferable and widely used as a pre-treatment for nucleic acid amplification. In these methods of extraction using silica-gel particle columns, however, the proteolytic enzyme also results in the degradation of an enzyme which is used for the amplification of a nucleic acid, and therefore it is required that after the treatment with the proteolytic enzyme and the treatments for nucleic acid extraction, the enzyme which is used for the amplification of the nucleic acid be added again, so as to carry out treatments for nucleic acid amplification, which causes a problem that many steps, including handlings, are necessary and it takes a lot of time.

In the above-described methods of pre-treatment in which the addition of neutralizing agents which are against substances inhibiting nucleic acid amplification, heating, and other treatments are carried out without separation of the nucleic acids, substances themselves in biological samples which inhibit nucleic acid amplification are not deactivated, which causes a problem that inhibitory effects by substances inhibiting nucleic acid amplification are not suppressed to a sufficient degree. In the above-described boiling methods, proteins can be deactivated by boiling treatments, but depending upon boiling conditions, are often not denatured to a sufficient degree, and the denaturing of proteins is unreliable.

An object of the present invention is to provide an enzyme-containing capsule which is employed for deactivating, from a sample containing proteins and nucleic acids, easily and simply the proteins in the sample, and then amplifying a nucleic acid in the sample, a kit for the amplification of a nucleic acid which comprises the enzyme-containing capsules, and a method for the amplification of a nucleic acid which employs the enzyme-containing capsules.

### Means for Solving the Problems

The present inventors have intensively studied so as to achieve the above object and found that by including in advance into a capsule a heat-resistant enzyme which is employed for nucleic acid amplification, treatment with a proteolytic enzyme can be achieved, while protecting the heat-resistant enzyme, and that by heat treatment after the proteolysis, the capsule can be dissolved, thereby releasing the heat-resistant enzyme into a sample solution, and thus the present invention has been completed.

The enzyme-containing capsule according to the present invention has a composition as indicated below:

(1) an enzyme-containing capsule comprising a heat-resistant enzyme, which is enveloped by a non-proteinous material having a melting point of 60 to 95° C.;
(2) the enzyme-containing capsule according to (1) described above, wherein the non-proteinous material is a bio-degradable plastic;
(3) the enzyme-containing capsule according to (2) described above, wherein the bio-degradable plastic is aliphatic polyester or a copolymer thereof;
(4) the enzyme-containing capsule according to (3) described above, wherein the aliphatic polyester or a copolymer thereof is polycaprolactone having a melting point of 60° C. or polybutylene succinate adipate having a melting point of 95° C.;
(5) the enzyme-containing capsule according to (1) described above, wherein the heat-resistant enzyme included therein in a state where the enzyme is dissolved in an antifreeze solvent;
(6) the enzyme-containing capsule according to (1) described above, wherein the heat-resistant enzyme is an enzyme which is employed for the amplification of a nucleic acid;
(7) the enzyme-containing capsule according to (6) described above, wherein nucleotides which are employed for the amplification of the nucleic acid are further included into the capsule;
(8) the enzyme-containing capsule according to (6) described above, wherein primers which are employed for the amplification of the nucleic acid are further included into the capsule.

The kit for the amplification of nucleic acid according to the present invention has a construction as indicated below:

(9) a kit for the amplification of nucleic acid, comprising the enzyme-containing capsule according to any one of (6) to 8) described above and a proteolytic enzyme;
(10) a kit for the amplification of nucleic acid, comprising one or more containers used in amplification reactions of nucleic acid, wherein each of the containers containing the enzyme-containing capsule according to any one of (6) to (8) described above, and an enzyme reaction buffer containing a proteolytic enzyme;
(11) a kit for the amplification of nucleic acid, comprising one or more containers used in amplification reactions of nucleic acid, wherein each of the containers containing the enzyme-containing capsule according to any one of (6) to (8) described above, and a capsule having a proteolytic enzyme and an enzyme reaction buffer included therein with an envelope comprising a non-proteinous material having a melting point of 30 to 50° C.;
(12) the kit for the amplification of nucleic acid according to any one of (9) to (11) described above, wherein the heat-resistant enzyme is included into the capsule in a state where the heat-resistant enzyme is dissolved in an antifreeze solvent;
(13) the kit for the amplification of nucleic acid according to (10) or (11) described above, wherein the enzyme reaction buffer further comprises nucleotides which are employed for the amplification of the nucleic acid;
(14) the kit for the amplification of nucleic acid according to (10) or (11) described above, wherein the enzyme reaction buffer further comprises primers which are employed for the amplification of the nucleic acid;
(15) the kit for the amplification of nucleic acid according to any one of (9) to (11) described above, wherein the proteolytic enzyme is proteinase K.

The method for the amplification of nucleic acid according to the present invention has a constitution as indicated below:

(16) a method for the amplification of nucleic acid comprising the steps of placing into a container used in amplification reactions of nucleic acid, an enzyme-containing capsule according to any one of (6) to (8) described above, a sample which contains a nucleic acid to be amplified, and optionally reaction substrates, primers, and an enzyme reaction buffer; and performing an amplification reaction of the nucleic acid in the container;
(17) the method for the amplification of nucleic acid according to (16) described above, wherein the step of performing the amplification reaction of the nucleic acid comprises the steps of melting the envelope of the enzyme-containing capsule at a temperature of 60 to 100° C.; and performing the amplification reaction of the nucleic acid at a temperature of 55 to 100° C.;
(18) a method for the amplification of nucleic acid, comprising the steps of (a) mixing a sample which contains proteins and a nucleic acid to be amplified, an enzyme-containing capsule according to any one of (6) to (8) described above, a proteolytic enzyme, and optionally reaction substrates, primers, and an enzyme reaction buffer, thereby preparing a reaction solution; (b) heating the reaction solution at a temperature of 30 to 60° C. for a period of 0 to 15 minutes; (c) further heating the heated reaction solution at a temperature of 60 to 100° C. for a period of 0 to 15 minutes; and (d) performing an amplification reaction of the nucleic acid employing the reaction solution;
(19) the method for the amplification of a nucleic acid according to (18) described above, wherein the proteolytic enzyme is included into the inside of a capsule with an envelope comprising a non-proteinous material having a melting point of 30 to 50° C. and wherein the step (b) is carried out at a temperature of said melting point to 60° C.;
(20) the method for the amplification of a nucleic acid according to (18) described above, wherein in the step (b), the reaction solution obtained in the step (a) is heated to 45 to 55° C.;
(21) the method for the amplification of a nucleic acid according to (19) described above, wherein the proteolytic enzyme has been included into the envelope in a state where the proteolytic enzyme is dissolved in an antifreeze solvent.

### EFFECTS OF THE INVENTION

In the enzyme-containing capsule according to the present invention, the protection of a heat-resistant enzyme, which is employed for nucleic acid amplification, is brought about by including the heat-resistant enzyme into the capsule, and therefore melting of the capsule after treatment with a proteolytic enzyme allows the heat-resistant enzyme to be released outside the capsule, with deactivating the proteolytic enzyme, so that a series of treatments ranging from treatment with a proteolytic enzyme to treatment for nucleic acid amplification can be carried out in a single container for amplification reactions of nucleic acid, and amplification of nucleic acids can be rapidly performed. Very easy and simple amplification of nucleic acids can be achieved, especially by using the kit for the amplification of nucleic acid according to the present invention.

In the method for the amplification of nucleic acid according to the present invention, all the reagents which are necessary in the process ranging from treatment with a proteolytic enzyme to treatment for nucleic acid amplification have been dispensed in advance into single containers for amplification reactions of nucleic acid, and handlings, such as dispensing, adding of reagents, and the like, are not required somewhere in the course of the process, so that it is possible to remarkably reduce the possibilities of contamination, secondary infection in cases where infectious samples are used, and others. In addition, heat treatments for releasing the heat-resistant enzyme from the capsule allow the proteins in sample solutions to be deactivated, and therefore it could be expected that the proteins in sample solutions can be deactivated to an unprecedented and effective degree.

### BRIEF DESCRIPTION OF THE DRAWING

FIG. 1 schematically shows a band pattern which was obtained by agarose gel electrophoresis of post-PCR reaction solutions obtained in Example 1 and Comparative Example 1, followed by staining with ethidium bromide. In the FIGURE, “Example” represents a lane where the post-PCR reaction solution obtained in Example 1 was run, “Comparative Example” represents a lane where the post-PCR reaction solution obtained in Comparative Example 1 was run, and “M” represents a lane where markers were run. The arrow, A, indicates a band of 238 bp.

### DETAILED DESCRIPTION OF THE PREFERRED EMBODIMENTS

A nucleic acid in the present invention is a nucleic acid of which the amplification is desired, and is not limited specifically, if it can serve as the template in amplification reactions. The nucleic acid may be a DNA, an RNA, or a cDNA synthesized from an RNA using a reverse transcriptase. The nucleic acid may be derived from organisms, such as humans, or synthesized.

A sample in the present invention is not limited specifically, if it contains a nucleic acid, and may contain proteins as contaminants. Included in this definition are biological samples, such as blood and body fluids, cultures, such as cultured cells and media, and others.

The enzyme-containing capsule of the present invention is characterized in that the capsule has a heat-resistant enzyme included therein with an envelope comprising a non-proteinous material having a melting point of 60 to 95° C. Here, the melting point of a capsule (an envelope) in the present invention means a temperature at which the capsule dissolves by heat treatment, and the non-proteinous material means a substance which is not degraded by proteolytic enzymes, that is, a substance having resistance to proteolytic enzymes.

The enzyme-containing capsule of the present invention has a heat-resistant enzyme in a state where the enzyme is included in the inside of the capsule which comprises a non-proteinous material as the envelope component. In this state, therefore, even when proteolysis treatment is carried out on samples to which the enzyme-containing capsule of the present invention has been added, the heat-resistant enzyme contained in the enzyme-containing capsule of the present invention is not degraded or deactivated. That is, in the enzyme-containing capsule of the present invention, the capsule plays a role as a protective membrane which protects the heat-resistant enzyme against effects of a proteolytic enzyme. In addition, since the melting point of the non-proteinous material which forms the envelope is from 60 to 95° C., the enzyme-containing capsule of the present invention can be dissolved by heating. Therefore, heating of the samples after the proteolysis treatment allows the heat-resistant enzyme protected with the envelope to be easily released, with deactivation of the proteolytic enzyme. The released heat-resistant enzyme can be utilized in reactions for amplifying a nucleic acid in samples.

The envelope component of an enzyme-containing capsule of the present invention is not limited specifically, if it has a melting point of 60 to 95° C. and is of a non-proteinous material. By the fact that the envelope component has a melting point of 60 to 95° C., proteolytic enzymes usually employed can be deactivated upon dissolving the capsule. Additionally, it is preferable that the capsule have a melting point of 60 to 70° C., in cases where methods for the amplification of nucleic acid are ones which do not involve the step of denaturing a double-stranded nucleic acid into single-stranded nucleic acids, as in the NASBA method using RNA as the template. In these cases, conditions where the heat-resistant enzyme has heat resistance will be relaxed and there will be an increasing variety of heat-resistant enzymes which can be contained in the enzyme-containing capsule of the present invention. More preferably, the non-proteinous material in the enzyme-containing capsule of the present invention is a bio-degradable plastic which is resistant to proteolytic enzymes. Further preferably, such a bio-degradable plastic is of an aliphatic polyester or a copolymer thereof. Further preferably, such an aliphatic polyester or a copolymer thereof is polycaprolactone having a melting point of 60° C. or polybutylene succinate adipate having a melting point of 95° C. The type, concentration, and the like of the capsule component of an enzyme-containing capsule of the present invention can be appropriately determined so as to attain a desired melting point, taking into account the type of the proteolytic enzyme and heat-resistant enzyme used, and others. The capsule component may be made of a single non-proteinous material or a mixture of two or more non-proteinous materials.

In the enzyme-containing capsule of the present invention, it is preferable that a heat-resistant enzyme be included into the capsule in a state where the heat-resistant enzyme is dissolved in an antifreeze solvent. Dissolving of a heat-resistant enzyme in an antifreeze solvent allows the enzyme-containing capsule of the present invention to be stored at or below 0° C. without loss of the enzymatic activity of the heat-resistant enzyme within the capsule. Here, an antifreeze solvent means a solvent that does not freeze at or below the freezing point of water. The antifreeze solvent is not limited specifically, if the solvent is one which is usually employed for storage of enzymes and others, and can be appropriately determined, taking into account the type of heat-resistant enzymes and others. Such antifreeze solvents include, for example, glycerol, ethylene glycol, diethylene glycol, polyethylene glycol, and the like.

The heat-resistant enzyme which is contained in the enzyme-containing capsule of the present invention is not limited specifically, and preferably is an enzyme which is employed for the amplification of a nucleic acid. The amplification of a nucleic acid in the present invention is by methods in which a nucleic acid in a sample is preferably used as the template and the strand of bases are extended using the complementation of nucleotides, thereby amplifying the nucleic acid in the sample. Such methods include, for example, PCR (Polymerase Chain Reaction), NASBA (Nucleic acid Sequence Based Amplification), LAMP (Loop mediated isothermal amplification) (see, for example, Patent Document 11), ICAN (Isothermal and Chimeric primer-initiated Amplification of Nucleic acids) (see, for example, Patent Document 12), and other methods.

The heat-resistant enzyme which is employed for the amplification of a nucleic acid in the present invention (hereinafter referred to as a nucleic acid amplifying heat-resistant enzyme) is not limited specifically, if the enzyme is an enzyme having heat resistance which is used in amplification reactions of nucleic acid, usually under conditions at temperatures of 60° C. or higher. Such a nucleic acid amplifying heat-resistant enzyme includes, for example, a heat-resistant DNA polymerase, a heat-resistant RNA polymerase, a heat-resistant RNA nuclease, and the like. The concentrations of heat-resistant enzyme which is contained in the enzyme-containing capsule of the present invention can be appropriately determined, taking into account the type and enzymatic activity of the heat-resistant enzymes used, and others.

The enzyme-containing capsule of the present invention can be produced by any method, if the method is one by which a heat-resistant enzyme can be included in the inside of the capsule without loss of its enzymatic activity. For example, an enzyme-containing capsule of the present invention can be produced by employing the method disclosed in Patent Document 13 (entitled “Improvements On And Relating To Capsulation”). The shape and size of an enzyme-containing capsule of the present invention are not limited specifically, if the capsule has a shape and the like which allow it to be added into reaction containers.

The enzyme-containing capsule of the present invention may contain heat-resistant materials other than the heat-resistant enzyme contained in the capsule, unless they impair the activity of the heat-resistant enzyme. For example, when the enzyme-containing capsule of the present invention contains a nucleic acid amplifying heat-resistant enzyme, the capsule may contain, for example, other reagents which are employed for the amplification of a nucleic acid. In particular, it is preferable that the capsule contain nucleotides and primers which are employed for the amplification of a nucleic acid, because the fact that nucleotides and others are contained in advance in the capsule along with the heat-resistant enzyme allows their separate dispensing procedures to be eliminated.

The kit for the amplification of nucleic acid according to the present invention is characterized in that the kit has an enzyme-containing capsule of the present invention and a proteolytic enzyme. The proteolytic enzyme is not limited specifically, if the proteolytic enzyme is one which does not have heat resistance, and can be any enzyme which is usually employed for degradation of proteins. Such a proteolytic enzyme is preferably an enzyme having no heat resistance, which exhibits an optimal temperature in the enzyme activity of less than 60° C. Particularly preferable is Proteinase K, because this enzyme has a superior enzymatic activity of proteolysis, is used for various purposes, and is available with ease.

The kit for the amplification of nucleic acid according to the present invention may further comprise one or more containers for amplification reactions of nucleic acid, wherein each of the containers may contains reagents and others which are necessary for a reaction of proteolysis with the above-described proteolytic enzyme and for an enzymatic reaction with the heat-resistant enzyme which is contained in the above-described enzyme-containing capsule. For example, the proteolytic enzyme may be included in the kit in a state where the proteolytic enzyme is dissolved in an enzyme reaction buffer. It is preferable that the enzyme reaction buffer be one which is suitable both for a proteolysis reaction and for an enzymatic reaction with the heat-resistant enzyme, because after the proteolysis reaction is completed, the enzymatic reaction with the heat-resistant enzyme can be performed without the need for further adjustments of the pH, salt concentrations, and others of the reaction solution. In cases where there are great differences in the composition between buffers suitable for a proteolysis reaction and for an enzymatic reaction with the heat-resistant enzyme, a capsule can be prepared which contains salts and others for adjusting the composition of the buffer, whereby such a capsule has been added, along with the enzyme-containing capsule, to the enzyme reaction buffer containing the proteolytic enzyme, so that handlings for adjusting the reaction solution can be eliminated after the completion of the proteolysis reaction. On the other hand, in cases where there is a slight possibility of impairing the enzymatic activity of the heat-resistant enzyme within the enzyme-containing capsule, salts and others for adjusting the buffer can be also contained in the enzyme-containing capsule.

It is preferable that each of the containers for amplification reactions of nucleic acid contain an enzyme-containing capsule, a proteolytic enzyme, and an enzyme reaction buffer, which are in amounts necessary for proteolysis reaction and the subsequent reaction of amplification of a nucleic acid, because handlings for dispensing the enzyme-containing capsule and others can be eliminated. The amount of enzyme-containing capsule to be required in the reaction of amplification of a nucleic acid can be appropriately determined, taking into account the type and concentration of the nucleic acid amplifying heat-resistant enzyme within the capsule, the type and volume of the solvent used, the volume of reaction solution used in the reaction of amplification of the nucleic acid, and others. It is preferable that the volume of the enzyme-containing capsule be not more than 1/10 of the volume of reaction solution where the reaction of amplification of a nucleic acid is carried out, in order to prevent effects in which the envelope component and solvent of the capsule dissolved in the reaction solution influence the reaction of amplification of the nucleic acid.

In cases where the solvent within the enzyme-containing capsule of the present invention is an antifreeze solvent, the kit for the amplification of nucleic acid according to the present invention can be stored under freezing conditions below 0° C. or under lyophilized conditions. For example, it is possible that a kit for the amplification of nucleic acid according to the present invention be stored without loss of the enzyme activity for longer periods by freeze-storing the kit at or below 0° C. than by cold-storing the kit. In cases where the kit is stored under the freezing conditions, it is preferable that freezing and thawing be not repeated, in order to preserve the enzymatic activity of the proteolytic enzyme.

Similarly, the enzymatic activity of proteolytic enzyme can be preserved in a more effective manner by containing the enzyme in another capsule in a state where the enzyme is dissolved in an antifreeze solvent. It is preferable that the envelope component of this capsule into which a proteolytic enzyme is included be insoluble in the solvent and to the contents within the capsule and is a non-proteinous material having a melting point of 30 to 50° C. The melting point of this capsule being on the order of 30 to 50° C., that is, not higher than temperatures suitable for treatment of proteolysis with the proteolytic enzyme which is included in the capsule, allows the capsule to retain the state where the proteolytic enzyme is dissolved in the antifreeze solvent, while the capsule is stored in the freezing conditions, and makes it possible for the capsule to dissolve, thereby releasing the proteolytic enzyme into the reaction solution at temperatures suitable for proteolysis treatment. Such an envelope component includes, for example, a mixture of galactan including 3,6-anhydrogalactose, guar gum, and starch, and the like (see, for example, Patent Document 14).

For example, when the kit for the amplification of nucleic acid according to the present invention is a kit for the amplification of nucleic acid which carries enzyme-containing capsules containing a nucleic acid amplifying heat-resistant enzyme, it is preferable that such a kit be one which carries containers for amplification reactions of nucleic acid which have the enzyme-containing capsule in their inside, and an enzyme reaction buffer containing a proteolytic enzyme. More preferably, the enzyme reaction buffer is one which is suitable both for proteolysis reaction with the proteolytic enzyme and for enzymatic reaction with the nucleic acid amplifying heat-resistant enzyme. Particularly preferable is a kit that further comprises nucleotides and primers used for the amplification of a nucleic acid. Since nucleotides and primers are resistant to proteolytic enzymes and do not have particular effects on the proteolysis reaction, they may be contained in an enzyme reaction buffer or the enzyme-containing capsule. When a proteolytic enzyme is contained in a capsule, nucleotides and primers may be contained in the same capsule as that which contains the proteolytic enzyme.

The method for the amplification of nucleic acid according to the present invention is not limited specifically, if the method is a method for the amplification of nucleic acid which employs an enzyme-containing capsule of the present invention. The method of the present invention can be also employed in cases where proteins are either absent from or present in samples to be amplified. In the former case, a nucleic acid can be amplified by carrying out the step of placing into a container used in amplification reactions of nucleic acid, an enzyme-containing capsule of the present invention, a sample which contains a nucleic acid to be amplified, and optionally reaction substrates, primers, and an enzyme reaction buffer, and then combining the step of performing an amplification reaction of a nucleic acid in the container. More specifically, the step of performing the amplification reaction of the nucleic acid can be a step of melting the envelope of the enzyme-containing capsule at a temperature of 60 to 100° C. for a period of 0 to 15 minutes, followed by performing the amplification reaction of the nucleic acid at a temperature of 55 to 100° C. When proteins are present in samples to be amplified, on the other hand, a nucleic acid in a sample can be amplified after the proteins in the sample are deactivated easily and simply by the method for the amplification of nucleic acid according to the present invention. For example, a nucleic acid in a sample which contains proteins and nucleic acids can be amplified as follows.

At first, as step (a), a sample which contains proteins and nucleic acids, an enzyme-containing capsule of the present invention, a proteolytic enzyme, and optionally reaction substrates, primers, and an enzyme reaction buffer are mixed, thereby preparing a reaction solution. At this time, it is preferable to add all the reagents that are necessary not only for the proteolysis reaction, but also for the subsequent reaction of amplification of a nucleic acid, such as nucleotides and primers to be employed for the amplification of the nucleic acid, and others. By adding and mixing in advance the reagents which are necessary in all the steps, handlings of addition of reagents are eliminated which are required somewhere in the course of the process and the convenience and quickness of procedures are enhanced, whereby the possibilities of contamination and secondary infection can be reduced. Nucleotides and primers may be added into and dissolved in reaction solutions, or added into reaction solutions in a state where they are contained in the enzyme-containing capsule along with a nucleic acid amplifying heat-resistant enzyme. The reagents that are employed for the proteolysis reaction and nucleic acid amplification reaction, such as enzyme reaction buffers, nucleotides, primers, and others, are not limited specifically, and those reagents which are usually employed when amplification of nucleic acids is carried out can be employed in amounts usually used.

Next, as step (b), the reaction solution obtained in the step (a) is heated at a temperature of 30 to 60° C. for a period of 0 to 15 minutes. Heating of the reaction solution at temperatures suitable for treatment of proteolysis with the proteolytic enzyme can lead to effective degradation of the proteins contained in the sample. In some embodiments, the proteolytic enzyme may be contained within a capsule having as the envelope component a non-proteinous material (having a melting point of 30 to 50° C.). In these cases, the heat treatment in the step (b) results in dissolving of the envelope component, so that the proteolytic enzyme acts on the proteins in the sample. For example, when proteinase K is used as the proteolytic enzyme, it is particularly preferable that the reaction solution obtained in the step (a) be heated to 45 to 55° C., because the enzymatic activity of proteinase K can be maintained at high levels.

As step (c), the reaction solution obtained in the step (b) is heated at a temperature of 60 to 100° C. for a period of 0 to 15 minutes after the proteolysis reaction is completed. This heat treatment allows the enzyme-containing capsule to dissolve, thereby releasing the nucleic acid amplifying heat-resistant enzyme into the reaction solution. In addition, since this heat treatment leads to denaturation and deactivation of the proteolytic enzyme, the released nucleic-acid-amplifying heat-resistant enzyme can be employed for the amplification of a nucleic acid without degradation of the released enzyme. Further, this heat treatment can also lead to denaturation of proteins contained in the sample which do not have heat resistance. That is, in the method for the amplification of nucleic acid according to the present invention, the proteins in a sample which are factors inhibiting amplification reactions of nucleic acid can be effectively deactivated by enzyme and heat treatments.

Further, as step (d), the reaction solution obtained in the step (c) can be employed to perform the amplification of a nucleic acid, so as to amplify a nucleic acid of interest in the sample. The amplification of a nucleic acid can be performed using the nucleic acid amplifying heat-resistant enzyme, by means of methods which are usually carried out. Reaction conditions and the like of the reaction of amplification of a nucleic acid can be appropriately determined, taking into account the length of a nucleic acid to be amplified, the type of primers, and others.

### EXAMPLES

### Example 1

The present invention is explained in more detail by way of Examples, but is not limited to the following Examples.

Enzyme-containing capsules containing DNA polymerase were prepared using polycaprolactone (a melting point of 60° C.) as an envelope component and employing the procedures described in Patent Document 13. Specifically, polycaprolactone was softened by partially solvating the polycaprolactone with N-methyl pyrrolidone and 1 μL of DNA polymerase solution to be included (1 unit) was supplied from a reservoir to prepare an enzyme-containing capsule 1. The DNA polymerase solution used a solution of DNA polymerase KODplus in 50% glycerol (1 unit; TOYOBO Co., Ltd.).

Then, PCR was performed using anticoagulated (EDTA-2K added) human blood (anticoagulated whole blood) as a sample and the resultant enzyme-containing capsule 1. Specifically, primer 1 having a base sequence of SEQ ID NO:1 and primer 2 having a base sequence of SEQ ID NO:2 were employed and a housekeeping gene, GAPDH (glyceraldehyde-3-phosphate dehydrogenase) gene, was used as the template to amplify a nucleic acid fragment of 238 bp.

At first, into a PCR tube were added one enzyme-containing capsule 1, 5 μL of 10× enzyme reaction buffer (200 mM Tris-HCl, 500 mM KCl, 80 mM MgCl₂), 0.5 μL of dNTPs (20 mM), 1 μL of primer 1 (15 μM), 1 μL of primer 2 (15 μM), 1 μL of proteinase K (20 mg/mL), and 40.5 μL of sterilized pure water and they were mixed, followed by adding 1 μL of anticoagulated whole blood, to prepare a reaction solution.

Then, the reaction solution was heated at 50° C. for 10 minutes. The reaction solution was further heated at 94° C. for 5 minutes, and then subjected to 30 rounds of a thermal cycle of 94° C. for 15 seconds, 55° C. for 30 seconds, and 68° C. for 30 seconds to perform PCR. The post-PCR reaction solution was subjected to agarose gel electrophoresis and then stained with ethidium bromide to ascertain whether or not the 238-bp nucleic acid fragment of interest had been amplified.

### Comparative Example 1

Instead of the enzyme-containing capsule 1, 1 μL of DNA polymerase KODplus (1 unit; TOYOBO Co., Ltd.) was used to amplify the 238-bp nucleic acid fragment of the GAPDH gene in a similar way as in Example 1. Specifically, the amplification was carried out as follows.

At first, into a PCR tube were added 1 μL of DNA polymerase KODplus, 5 μL of 10× enzyme reaction buffer (200 mM Tris-HCl, 500 mM KCl, 80 mM MgCl₂), 0.5 μL of dNTPs (20 mM), 1 μL of primer 1 (15 μM), 1 μL of primer 2 (15 μM), and 41.5 μL of sterilized pure water and they were mixed, followed by adding 1 μL of anticoagulated whole blood, to prepare a reaction solution.

Then, the reaction solution was heated at 50° C. for 10 minutes. The reaction solution was further heated at 94° C. for 5 minutes, and then subjected to 30 rounds of a thermal cycle of 94° C. for 15 seconds, 55° C. for 30 seconds, and 68° C. for 30 seconds to perform PCR. The post-PCR reaction solution was subjected to agarose gel electrophoresis and then stained with ethidium bromide to ascertain whether or not the 238-bp nucleic acid fragment of interest had been amplified.

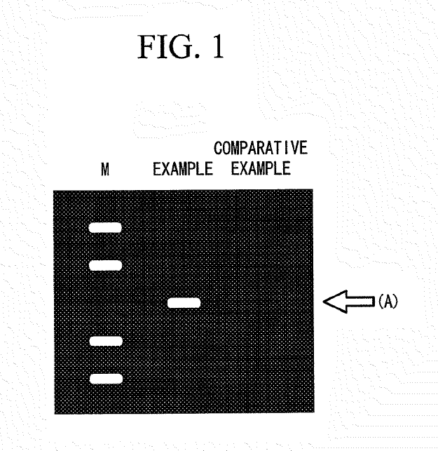

FIG. 1 schematically shows a band pattern which was obtained by agarose gel electrophoresis of post-PCR reaction solutions obtained in Example 1 and Comparative Example 1, followed by staining with ethidium bromide. In the FIGURE, “Example” represents a lane where the post-PCR reaction solution obtained in Example 1 was run, “Comparative Example” represents a lane where the post-PCR reaction solution obtained in Comparative Example 1 was run, and “M” represents a lane where markers were run. The arrow, A, indicates a band of 238 bp. As is clear from FIG. 1, the 238-bp nucleic acid fragment of interest was found to have been amplified in the post-PCR reaction solution obtained in Example 1, whereas no amplified nucleic acid fragment was detectable in Comparative Example 1. It is inferred that this observation was due to the fact that unlike Example 1, Comparative Example 1 had not eliminated proteins which were substances inhibiting the amplification, because in Comparative Example 1, the addition of proteinase K to the reaction solution resulted in the deactivation of the DNA polymerase. From the finding that the amplified nucleic acid fragment was detected in Example 1, it is apparent that the enzyme-containing capsule of the present invention is not affected by proteolytic enzymes, such as proteinase K, and that the DNA polymerase is released into the reaction solution by the same heat treatment (at 94° C. for 5 minutes) as in a denaturing step which is usually employed in PCR, so that PCR is performed.

From these results, it is clear that by employing the enzyme-containing capsule of the present invention, proteolysis treatment of biological samples, such as whole blood, can be carried out with no effect on the enzymatic activity of heat-resistant enzymes, such as DNA polymerases, and reaction solutions for proteolysis reaction and for the subsequent amplification reaction of a nucleic acid can be prepared at a time, thereby allowing a nucleic acid in a sample to be rapidly amplified.

### Example 2

First, 1 μL of DNA polymerase solution (1 unit) was prepared in a similar way as in Example 1.

Enzyme-containing capsules containing DNA polymerase were prepared using polycaprolactone (a melting point of 60° C.) as an envelope component and employing the procedures described in Patent Document 13. Specifically, polycaprolactone was softened by partially solvating the polycaprolactone with N-methyl pyrrolidone and 3.5 μL of enzyme solution to be included was supplied from a reservoir to prepare an enzyme-containing capsule 2. The enzyme solution was a mixed solution containing, in 3.5 μL, 1 μL of the DNA polymerase solution (1 unit), 0.5 μL of dNTPs (20 mM), 1 μL of primer 1 (15 μM), and 1 μL of primer 2 (15 μM).

Instead of the enzyme-containing capsule 1, the enzyme-containing capsule 2 was used to amplify the 238-bp nucleic acid fragment of the GAPDH gene in a similar way as in Example 1. Specifically, the amplification was carried out as follows.

At first, into a PCR tube were added one enzyme-containing capsule 2, 5 μL of 10× enzyme reaction buffer (200 mM Tris-HCl, 500 mM KCl, 80 mM MgCl₂), 1 μL of proteinase K (20 mg/mL), and 40.5 μL of sterilized pure water and they were mixed, followed by adding 1 μL of anticoagulated whole blood, to prepare a reaction solution.

Then, the reaction solution was heated at 50° C. for 10 minutes. The reaction solution was further heated at 94° C. for 5 minutes, and then subjected to 30 rounds of a thermal cycle of 94° C. for 15 seconds, 55° C. for 30 seconds, and 68° C. for 30 seconds to perform PCR. The post-PCR reaction solution was subjected to agarose gel electrophoresis and then stained with ethidium bromide to ascertain whether or not the 238-bp nucleic acid fragment of interest had been amplified.

Based on the observation of this experiment, it could be ascertained that as in the post-PCR reaction solution obtained in Example 1, the nucleic acid fragment of 238 by had been amplified.

### Example 3

Capsules containing proteinase K (hereinafter referred to as proteinase-K-containing capsules) were prepared using a mixture of a galactan composite, guar gum, and starch (galactan mixture; a melting point of 50° C.) as an envelope component and employing the procedures described in Patent Document 14. The galactan mixture was a mixture in which a galactan composite, guar gum, and starch were at a ratio of 100:33:133 by weight. The galactan composite was a composite of a first galactan and a second galactan, the first galactan having a molecular weight of 300,000 to 700,000 and a 3,6-anhydrogalactose content of 20 to 30% by weight, and the second galactan having a molecular weight of 3,000 to 250,000 and a 3,6-anhydrogalactose content of 30 to 40% by weight, in which the second galactan was at a ratio of 60 parts by weight, relative to 100 parts by weight of the first galactan. Specifically, to the above-mentioned mixture, 1 μL of proteinase K solution (20 mg/mL) to be included was supplied from a reservoir employing a rotary-die-typed filling machine, to produce a proteinase-K-containing capsule. A solution where 1 μL of proteinase K (40 mg/mL) was dissolved in 1 μL of 100% glycerol was used as the proteinase K solution.

The enzyme-containing capsule 1 obtained in Example 1 and the proteinase-K-containing capsule were employed to amplify the 238-bp nucleic acid fragment of the GAPDH gene in a similar way as in Example 1. Specifically, the amplification was carried out as follows.

At first, into a PCR tube were added one enzyme-containing capsule 1, one proteinase-K-containing capsule, 5 μL of 10× enzyme reaction buffer (200 mM Tris-HCl, 500 mM KCl, 80 mM MgCl₂), 0.5 μL of dNTPs (20 mM), 1 μL of primer 1 (15 μM), 1 μL of primer 2 (15 μM), and 40.5 μL of sterilized pure water and they were mixed, followed by adding 1 μL of anticoagulated whole blood, to prepare a reaction solution.

Then, the reaction solution was heated at 50° C. for 10 minutes. The reaction solution was further heated at 94° C. for 5 minutes, and then subjected to 30 rounds of a thermal cycle of 94° C. for 15 seconds, 55° C. for 30 seconds, and 68° C. for 30 seconds to perform PCR. The post-PCR reaction solution was subjected to agarose gel electrophoresis and then stained with ethidium bromide to ascertain whether or not the 238-bp nucleic acid fragment of interest had been amplified.

Based on the observation of this experiment, it could be ascertained that as in the post-PCR reaction solution obtained in Example 1, the nucleic acid fragment of 238 by had been amplified. From this result, it is clear that the heat treatment at 50° C. allowed the proteinase-K-containing capsule to dissolve, thereby releasing the proteinase K included therein into the reaction solution, such that effective deactivation of the proteins in the sample is achieved. Therefore, it is clear that by using the enzyme-containing capsule of the present invention and a capsule having a proteolytic enzyme contained therein, rapid amplification of a nucleic acid in a sample can be also carried out.

### INDUSTRIAL APPLICABILITY

By using the enzyme-containing capsule of the present invention, effective deactivation of the proteins in samples can be achieved employing a proteolytic enzyme under conditions where a heat-resistant enzyme has been added, and therefore it is possible to utilize the enzyme-containing capsule of the present invention, particularly in the field of gene analysis and the like using biological samples.

1. A composition for nucleic acid amplification comprising:
a capsule;
a heat-resistant enzyme for nucleic acid amplification contained within the capsule;
wherein the capsule is resistant to proteolytic enzymes and wherein the capsule protects the heat-resistant enzyme from proteolysis.

2. The composition of claim 1 wherein the capsule in a dissolved state releases the heat-resistant enzyme.

3. The composition of claim 2 wherein the capsule has a melting point and the capsule enters the dissolved state when the capsule temperature is greater than the melting point temperature.

4. The composition of claim 3 wherein the melting point is about 60° C. to about 95° C.

5. The composition of claim 1 wherein the capsule includes a bio-degradable plastic.

6. The composition of claim 5 wherein the bio-degradable plastic includes an aliphatic polyester.

7. The composition of claim 5 wherein the bio-degradable plastic includes a polycaprolactone or a polybutylene succinate adipate.

8. The composition of claim 1 wherein the heat-resistant enzyme is dissolved in an anti-freeze solvent.

9. The composition of claim 8 wherein the anti-freeze solvent includes glycerol, ethylene glycol, diethylene glycol, or polyethylene glycol.

10. The composition of claim 1 wherein the heat-resistant enzyme for nucleic acid amplification includes a heat-resistant DNA polymerase.

11. The composition of claim 1 further comprising nucleotides for nucleic acid amplification contained within the capsule.

12. The composition of claim 1 further comprising a primer for nucleic acid amplification contained within the capsule.

13. A method of amplifying a nucleic acid from a sample which contains a protein and a nucleic acid, the method comprising the steps of:
combining the sample, a proteolytic enzyme, and the composition of claim 3 to produce a reaction mixture;
maintaining the reaction mixture under conditions consonant with proteolysis by the proteolytic enzyme; and
releasing the heat-resistant enzyme within the capsule to combine the heat-resistant enzyme with the sample by raising the temperature of the reaction mixture above the melting point of the capsule.

14. The method of claim 13 wherein the step of maintaining the reaction mixture under conditions consonant with proteolysis by the proteolytic enzyme includes heating the reaction mixture to a temperature less than the melting point of the capsule.

15. The method of claim 14 wherein the step of maintaining the reaction mixture under conditions consonant with proteolysis by the proteolytic enzyme includes heating the reaction mixture to about 30° C. to about 60° C. for 0 to 15 minutes.

16. The composition of claim 13 wherein the proteolytic enzyme is deactivated at temperatures above the melting point.

17. The method of claim 13 wherein the proteolytic enzyme includes proteinase K.

18. A kit for the amplification of a nucleic acid comprising the composition of claim 1 and a proteolytic enzyme.

19. The kit of claim 18 wherein the proteolytic enzyme includes proteinase K.

20. The kit of claim 18 further comprising a substrate for the heat-resistant enzyme and an amplification primer.
